(19) United States
(12) Patent Application Publication
(10) Pub. No.: US 2004/0067560 A1
(43) Pub. Date: Apr. 8, 2004

(54) Enzymatically modified hydrophobic starch

(75) Inventors: Helene G. Bazin, Cincinnati, OH (US); Frank W. Barresi, Coralville, IA (US); Jiao Wang, Muscatine, IA (US)

Correspondence Address: BANNER & WITCOFF, LTD., TEN SOUTH WACKER DRIVE, SUITE 3000, CHICAGO, IL, 60606, US

(73) Assignee: Grain Processing Corporation, Muscatine, IA

(21) Appl. No.: 10/679,668

(22) Filed: Oct. 6, 2003

Related U.S. Application Data
(63) Continuation of application No. 09/961,867, filed on Sep. 24, 2001, now abandoned.
Application No. 09/961,867 is a continuation-in-part of application No. 09/667,355, filed on Sep. 22, 2000, now abandoned.

(51) Int. Cl. C12P 19/04; C08B 31/00

(52) U.S. Cl. 435/101; 536/102

(57) ABSTRACT

Disclosed are starch granules prepared via treating starch with a glucoamylase enzyme. The starch granules prepared in accordance with the present invention are hydrophobic relative to native starch granules, and are suitable for use in numerous applications. Also disclosed are a porous starch product, a delayed release product, and a method for absorbing fluid from the skin. The delayed release product comprises a product carried within the pores of the porous granular starch. The method for absorbing fluid comprises applying a fluid-absorbing effective amount of the dried, ground granules.

### RELATED APPLICATION

This application is a continuation-in-part of prior U.S. application Ser. No. 09/667,355, the entire contents at which are hereby incorporated by reference.

### TECHNICAL FIELD OF THE INVENTION

The invention is in the field of starch derivatives, and more specifically pertains to the enzymatic modification of granular starches to result in a hydrophobic starch product.

### BACKGROUND OF THE INVENTION

Enzymes capable of hydrolyzing granular starch at temperatures below the starch gelatinization temperature are known in the art. For instance, it has long been known that alpha-amylases can hydrolyze granular starch, as disclosed in, for instance, Richert et al., Publication of the Carnegie Institution at Washington, No. 173, Part 1 (1913). More recently, other enzymes, such as glucoamylase enzymes, have also been found to hydrolyze granular starch below the starch gelatization temperature. It is believed that the presence of a starch-binding domain is essential for an enzyme to hydrolyze granular starch; numerous enzymes having such domains are known, as disclosed, for instance, in Walker, G. J. et al. Biochemical Journal, 86:452 (1963); Belshaw, N. J. et al., Biochim Biophys Acta, 1078:1117-20 (1991), and Svensson, B. et al., Eur. J. Biochem, 154:497-502 (1986).

As is also known in the art, when a granular starch is treated with an alpha amylase or a glucoamylase, the granular structure of the starch degrades, leaving behind a porous starch granule upon partial hydrolysis of the starch, or, if the enzymatic hydrolysis is allowed to continue, yielding a starch hydrolyzate or ultimately glucose or another lower order sugar. It is also recognized that the enzymatic attack on starch granules takes place by exo-corrosion in which the enzyme either erodes the entire surface of the granule or digests a channel from points on the surface towards the center of the granule. In the latter mode of attack, once the center is reached, the enzymatic attack proceeds outwardly from the center over a broader front. The internal structure of a porous starch granule that has been so modified is open and cavernous and can exhibit either a terraced or a step-shaped appearance.

When a glucoamylase enzyme is allowed to completely hydrolyze a starch granule, the resulting product typically is glucose. U.S. Pat. Nos. 2,583,451; 3,922,198; 3,922,199; 4,612,284; and 4,618,579 disclose processes for converting granular starch to glucose by treating of the starch with glucoamylase or a mixture of glucoamylase with alpha-amylase. Other reaction products are possible; for instance, U.S. Pat. No. 3,922,201 discloses a process for the preparation of levulose-containing compositions from granular starch by treating the starch with alpha-amylase, glucoamylase, and glucose isomerase.

The prior art also has described the enzymatic hydrolysis of starch below the gelatinization temperature to produce starch hydrolyzates other than glucose. For instance, U.S. Pat. No. 3,922,196 discloses a process for converting granular starch to a starch hydrolyzate having a DE (dextrose equivalent) between 40 and 55 and including a high percentage of disaccharides and trisaccharides. The process disclosed in this patent employs alpha-amylase, glucoamylase, beta-amylase and isoamylase. Another document, U.S. Pat. No. 4,113,509, discloses an enzymatically produced high maltose-maltotriose starch hydrolyzate having a DE of 40 to 55. This patent discloses a process in which alpha-amylase, alone or with a saccharifying enzyme such as glucoamylase or beta-amylase, is used to hydrolyze the starch. Methods for the production of other malto-oligosaccharides such as maltose and maltotetraose by treatment of starch with specific alpha-amylases have also been employed on an industrial scale.

The prior art also has provided applications for porous starches that are obtained by partial enzymatic digestion of the granular starch. For instance, U.S. Pat. No. 4,985,082 discloses a starch matrix material comprising granular starch that is partially hydrolyzed with an alpha-amylase and/or a glucoamylase and treated chemically to modify the structural integrity and surface characteristics of the starch. The disclosed starches are said to be useful as adjuvants for antiperspirants and as bulking agents for foods and drinks. U.S. Pat. No. 4,551,177 discloses a compressible starch said to be useful as a binder for a tablet or capsule and which is said to be prepared by treating granular starch with an acid and/or with an alpha-amylase enzyme at a temperature below the gelatinization temperature of the starch. Another document, EP 182,296 discloses a body dusting powder that comprises a porous starch granule which consists essentially of the residue remaining after about from 45% to 95% by weight of the granular starch has been solublized with an enzyme. Yet another document, U.S. Pat. No. 5,445,950, discloses a method of using alpha amylase to prepare slightly decomposed starch granules having low viscosity. The starch granules are said to be useful as a raw material in the starch and sugar industry. U.S. Pat. No. 5,904,941 discloses a viscosifier that comprises an enzymatically hydrolyzed, ungelatinized granular starch with a dextrose equivalent of from about 5 to 60. Still another document, U.S. Pat. No. 5,935,826, discloses a modified starch prepared by the glucoamylase hydrolysis of a starch derivative that contains a hydrophobic group or both a hydrophobic and a hydrophilic group. The starches are said to be characterized by having a DE from 20 to 80, and are said to be useful as emulsifiers or an encapsulating agents. International Patent Publication WO 96/10586 discloses a method for preparing a fat substitute based on hydrolyzed granular starch. U.S. Pat. No. 5,919,486 discloses a powder preparation that comprises a porous starch grain carrier and a material carried within the pores of the carrier, the porous starch grain carrier having been prepared by partially hydrolyzing starch with raw starch digestive enzyme.

The prior art discussed above does not describe starch granules that are hydrophobic (i.e. substantially more resistant to wettability) relative to starch that has not been enzymatically modified. Wettability in this context refers to the tendency of water or other aqueous media to wet the surface of the starch granule. As set forth in more detail hereinbelow, this property can be evaluated by observing the properties of the starch granules in aqueous suspension. A hydrophobic granular starch would be useful in connection with a number of applications such as cosmetics and other personal care products, pharmaceutical products and food and industrial products, especially where properties such as grease mitigation are required. It is thus a general object of the present invention to provide a hydrophobic granular starch.

### THE INVENTION

It has now been discovered that the treatment of unmodified or cross-linked granular starches with glucoamylase in aqueous solution, preferably alone but optionally in combination with relatively smaller amounts of other enzymes, followed by the lowering of the pH of the solution, surprisingly yields a starch granule that is highly hydrophobic relative to starch granules that have not been treated. It has been found preferable to avoid hydrolyzing the starch to any significant extent if the hydrophobic properties of the starch are to be maximized. If the starch is hydrolyzed to provide a porous starch, substances can be readily absorbed into the porous granules thus prepared to provide a product that remains flowable and in powder form. A porous starch granule thus prepared also exhibit an initial hydrophobic character, such that water will not pass through a layer of the granules on initial contact. After more prolonged contact, the porous starch granules will exhibit improved absorption of water and saline relative to non-hydrolyzed starch granules. The starch granules thus not only are useful in connection with delayed release applications such as for flavors, fragrances, and the like but also are useful in connection with other applications, such as skin care applications. In highly preferred embodiments of the invention, the starch is not hydrolyzed, or is hydrolyzed only minimally. In the embodiments the hydrophobic starch granules are extremely resistant to wet-out, and have an affinity for oleogenous materials. Such granules are useful in conjunction with numerous cosmetic and personal care applications and other applications.

Thus, in accordance with the invention, a method is provided for preparing hydrophobic starch granules. Generally, the method comprises treating the starch granules with a glucoamylase enzyme in aqueous solution at a temperature below the gelatinization temperature of the starch and lowering the pH of the solution to a level effective to render the surface of the starch granule hydrophobic. The enzymatic reaction should be terminated before the starch granules are completely hydrolyzed and preferably before any hydrolysis has occurred. The invention also encompasses the granular starch product prepared thereby. In some embodiments, the invention encompasses a product that comprises a material carried in the pores of the starch.

Even further, the invention encompasses a method for absorbing fluids from the skin, the method comprising applying an amount of the starch granules effective for this purpose.

Other features and embodiments of the invention are described hereinbelow.

### DESCRIPIION OF PREFERRED EMBODIMENT

Generally, the invention contemplates the treatment of a granular starch with a glucoamylase enzyme or with another enzyme or sequence of amino acids that has an effect that is comparable to those of the enzymes described herein. The starches which may be used as starting materials in connection with the invention may be derived from any native source, and typical starch sources include cereals, tubers, roots, legumes, and fruits. Exemplary starches include those obtained from corn, potato, wheat, rice, sago, tapioca, and sorghum. In many embodiments, the starch preferably is corn starch, but other starches, such as high amylose starches, may also be used in conjunction with the invention and may be preferred in some applications. Suitable starches include pearl starches, such as PURE-DENT® B700 and corn starch B200 sold by Grain Processing Corporation of Muscatine, Iowa. The starch used in conjunction with the invention not only may be a native starch, but also may be a starch that has been modified prior to enzymatic hydrolysis. Exemplary of such modified starches are cross-linked starches, which may comprise a native starch that has been cross-linked via any suitable cross-linking technique known in the art or otherwise found to be suitable in conjunction with the invention. An example of a commercially available cross-linked starch is PURE-DENT® B850, sold by Grain Processing Corporation of Muscatine, Iowa. Other starches are suitable for use in conjunction with the invention, and thus it is contemplated that, for instance, derivatized, acid-thinned, or otherwise modified starches may be employed. In some embodiments, a non-granular starch that comprises dried, ground pregelatinized starch may be employed as a starting material. Such starches should be deemed to be granular starches within the purview of the invention.

In accordance with the invention, the starch is treated with a glucoamylase enzyme or with another enzyme or sequence of amino acids. Suitable enzymes for use in conjunction with the invention are believed to include any of a wide variety of glucoamylases, and include those derived from fungal, bacterial, or animal origin. Glucoamylases are known to remove glucose units in a stepwise manner from the non-reducing end of the starch and to cleave both 1-4 and 1-6 linkages in the starch molecule. Preferred glucoamylases include those derived from Aspergillus niger, other glucoamylase enzymes have been found largely ineffective. One glucoamylase suitable for use in conjunction with the invention is G990, a glucoamylase enzyme that is commercially available from Enzyme Biosystems Ltd. It is known in the literature that the glucoamylase enzyme includes a starch binding domain. It is now further believed that the glucoamylase enzyme includes other regions that are responsible for exposing a hydrophobic “surface” when the pH of the surrounding solution is lowered to a level effective to denature the enzyme. It is contemplated that enzymes other than glucoamylase that are capable of binding to or otherwise associating with the starch granule and that are capable of exposing the hydrophobic “surface” may be employed in addition to or in lieu of the glucoamylase enzyme. Non-enzymatic amino acid sequences also may be employed. The starch should be treated with the glucoamylase enzyme under conditions suitable to yield a hydrophobic starch granule. Generally, the enzymatic treatment is accomplished in an aqueous or buffered slurry at any suitable starch solids level, preferably a solids level ranging from about 10% to about 55% by weight on dry starch basis, more preferably about 25% to about 45% by weight. In other embodiments an enzyme solution may be applied to dry starch granules, or a dry enzyme may be applied to wet granules. In any event the enzyme will contact the starch in an aqueous enzyme solution. The pH and temperature of the slurry should be adjusted to any conditions effective to allow the enzyme hydrolysis to bind to or otherwise associate the starch granule. These will vary depending on the enzyme and starch selected, and are not critical so long as the starch does not gelatinize; generally, this can be accomplished so long as the temperature remains below the gelatinization temperature of the starch. In general, the pH will range from about 3.0 to about 7.5; more preferably, the pH should range from about 3.5 to about 6.0. To reach this pH, any suitable acid or base may be added, or a buffer may be employed. The temperature preferably is maintained at a temperature of at least 3° C. below the gelatinization temperature of the starch. For corn starch, the gelatinization temperature falls within a range between about 62° and 72° C. Accordingly, the temperature of the slurry should be below about 62° C., preferably ranging from about 22° C. to about 59° C., and more preferably from about 40° C. to about 55° C.

The glucoamylase may be employed in any amount suitable to effectuate a hydrophobic character of the starch granules in the slurry. Preferably, the glucoamylase is employed in the slurry in a concentration ranging from about 0.2% to about 6%, more preferably 0.4% to about 4% by weight on dry starch, and more preferably from about 1% to about 3%, based on a 300 unit per ml enzyme (based on the Enzyme Biosystem unit definition). Other enzymes may be used in conjunction with the glucoamylase in smaller amounts. For instance, endo-alpha-amylases, which cleave the 1-4 glucosidic linkages of starch; beta-amylases, which remove maltose units in a stepwise fashion from the non-reducing ends of the alpha-1,4-linkages; and debranching enzymes, such as iso amylase and pullulanse, which cleave 1-6 glucosidic linkages of the starch molecules, may be employed. Sources of alpha-amylases, beta-amylases, and pullulanses include, for instance, several species of the Bacillus microorganism, such as Bacillus subtilis, Bacillus licheniformis, Bacillus coagulans, Bacillus amyloliquefaciens, Bacillus stearothermophilus, and Bacillus acidopullulyticus When used, such other amylases should not be used in concentration higher than about 0.015%, by weight on dry starch (based on Enzyme Biosystems G995 enzyme), or, more generally, from about 0.5% to about 7.5% of the amount of glucoamylase enzyme. If too great a quantity of another enzyme is used, a conventional porous starch granule that lacks hydrophobic character will be produced. Thus, generally speaking, the other enzyme may be used in any amount effective to enhance the starch hydrolysis without destroying the hydrophobic property of the resulting starch granules. In the preferred embodiments no additional enzyme is employed.

In the highly preferred embodiments of the invention, the enzyme or amino acid sequence is allowed to bind to the starch granule, but (in the case of an enzyme) the enzyme is not allowed to hydrolyze the starch, or is allowed to hydrolyze the starch to as little an extent as possible. The enzyme preferably does not hydrolyze the starch to a greater extend than 5%, more preferably not more than 1%, before the enzymatic action is terminated. Generally the enzyme should be allowed to bind to the starch for 0.1-15 minutes to achieve this result.

In less preferred embodiments, the reaction may be allowed to proceed until the starch has been hydrolyzed to yield a porous granule. The starch granule should be hydrolyzed to a yield ranging from about 1% to about 50%, as may be evidenced by changes in the granular interior structure or surface structure when viewed under scanning electron microscopy, or by the properties of the resulting granules. Typically in such less preferred embodiments, it is contemplated that the enzymatic reaction will take from about 15 minutes to about 120 hours, more typically from about 2 hours to about 8 hours, depending upon the type of starch used, the amount of enzyme used, and other reaction parameters. It is contemplated that as a result of enzymatic cleavage of the starch molecule the porous granular body that remains may comprise oligosaccharides of lower molecular weight in addition to starch; such granular structure is still deemed to be a porous starch granule within the purview of the present invention.

When it is desired to terminate the enzymatic action, the enzymatic action may be terminated by any suitable techniques known in the art, including acid or base deactivation, ion exchange, solvent extraction, or other suitable techniques. Preferably, heat deactivation is not employed, since a granular starch product is desired and since the application of heat in an amount sufficient to terminate the enzymatic reaction may cause gelatinization of the starch. In any event, regardless of whether any additional terminating step is employed, the pH of the starch slurry is lowered to a level effective to denature the enzyme or said sequence and to render hydrophobic the surface of the starch granules. Generally this may be accomplished by lowering the pH to a value lower than 2.0 for at least 5 minutes, typically for 5 to 30 minutes. After deactivation, the pH of the slurry may be readjusted to the desired pH according to the intended end use of the granules. Typically, the pH will be adjusted to a pH within the range from about 5.0 to 7.0, more preferably from about 5.0 to about 6.0. The starch granules thus prepared then can be recovered using techniques known in the art, including filtration and centrifugation. Any reducing sugars and other byproducts produced during the enzymatic treatment may be removed during the washing steps. Most preferably, the starch granules subsequently are dried to moisture content of or below about 12%.

The hydrophobic starch produced in accordance with the preferred embodiments of the invention exhibit a strong hydrophobic property and a stronger affinity for oleogenous compounds such as greases, oils, and waxes than other starches. Such hydrophobic materials are more readily blended with the starch of the invention than with other starches to produce mixtures with a less greasy texture. Exemplary applications include baby and such powders, liquid talc, lotions, creams, ointments, sunscreens, color cosmetics, liquid and power makeup, mascaras, eyeliners, eye shadow, antiperspirants, processing aids for Vitamin E, anti-caking agents for foods and other products, dusting agents for gloves and other materials, coating agents (especially for water resistant coatings), flavor masking agents and so forth. Many of these embodiments employ a skin contacting agent (e.g. a color component, body agent, cream base etc.) and an amount of the starch of the invention effective to absorb oil from the skin when the product is applied to the skin.

Embodiments of the invention in which the enzyme has been allowed to hydrolyze the starch to 5% or greater are less preferred, but nonetheless yield starch granules that are useful in numerous applications. The starch granules thus prepared may be used as a carrier matrix for a product such as a flavor, fragrance, or the like. In accordance with this aspect of the invention, a carried product, such as a carried flavor or fragrance, may be prepared by contacting the porous starch granules with a material in an amount effective to cause at least some of the material to become carried within the pores formed by the enzymatic hydrolysis, such as by mixing the granules with a liquid that contains the material and allowing the material to become absorbed into the pores. The material may be a water-soluble material, or may be a material that is not water-soluble (for instance, a fragrance oil). In another embodiment, the dried starch granules may be ground, and used as an absorber. For instance, it has been found that dried, ground starch granules prepared in accordance with such embodiments are suitable for use in absorbing moisture and oils from the skin. The dried, ground product thus is suitable for use in connection with deodorants, facial creams, baby powders, and other skin care products. The invention thus encompasses a method for absorbing fluid from the skin, the method including the step of applying a fluid-absorbing effective amount of the porous starch product, which preferably is the dried, ground product. The fluids that may be absorbed from the skin include water-based fluids, (such as sweat) and oil-based fluids, and include natural skin fluids as well as fluids that have been applied to the skin. Alternatively, the dried ground granules may be contacted with a flavor, fragrance, or other material, and the product thus formed may be used in any suitable application.

The starch granules prepared in accordance with such less preferred embodiments of the invention typically display a mix of unique properties, including enhanced water and saline absorption properties. It has been found that unlike conventional porous starches that have lower density and larger surface area than non-porous granules, the dried bulk density of the starch granules of the invention is approximately the same as that of native starch granules, and the surface area of the starch granules is slightly increased relative to native starch granules when glucoamylase alone is used. While it is not intended to limit the invention to a particular theory of operation, it is believed that the hydrophobic properties and delayed aqueous wettability are more likely the result of a chemical change at the surface of the starch granule than a physical change, in which entrapped air would explain the properties of the starch granule.

The following Examples are presented to further illustrate the invention and should not be viewed as limiting the invention.

### EXAMPLES

The following protocols were used to evaluate the starch granules prepared in accordance with the invention and the comparative examples.

### Hydrophobicity

Prior to testing, each sample subject to evaluation was screened through a 325 mesh (US) screen (0.0045 cm, 0.0017 in.). Into a 150 ml beaker was poured 1100 ml distilled water, and 2.0 g of the sample were sprinkled on top of the water. A finger was stuck into the beaker below the surface of the water, and immediately withdrawn. If the finger was dry, the sample was deemed to exhibit hydrophobicity character; if the finger was wet, the sample was not deemed to exhibit hydrophobicity.

### Delayed Wettability

Delayed wettability provides another qualitative measure of the hydrophobic nature of the starch granules. Into a 150 ml beaker was poured 1100 ml distilled water, and 5.0 g of the sample were sprinkled on top of the water. This mixture was mechanically stirred with a spatula. If the starch formed a suspension in the water in the same amount of time as the unhydrolyzed starch, the test was deemed negative. If the starch did not readily form a suspension, but rather stayed on the surface of the water before forming a suspension, the sample was deemed to exhibit delayed wettability.

### Water, Saline, and Oil Absorption

Prior to testing, each sample was screened through a 120 mesh (US) screen (0.0125 cm, 0.0049 in). Absorption was evaluated in accordance with ASTM D281-95, a standard test method for oil absorption of pigments by spatula rub-out. To perform the test, the absorption solution (water, a 1% saline solution, or a mineral oil (CHEVRON SUPERLA #7)) was added dropwise to 10.0 g (dry solids basis) starch (weighed in a 100 ml beaker) until a stiff, putty-like paste was formed; the amount of fluid needed to reach this point was recorded as the test result. The precision of this test is +/−0.5 ml.

### Example 1

This Example illustrates the preparation of porous starch granules using glucoamylase.

Five hundred grams (dry solids basis) of dent corn starch were slurried in 1250 ml tap water. The slurry was heated to 60° C. and the pH adjusted to the value indicated in Table 1 using dilute hydrochloric acid. To the slurry was added the indicated amount of glucoamylase G990, a commercially available enzyme sold by Enzyme Bio-systems Ltd., and the reaction was allowed to proceed at the indicated temperature with constant mixing for the indicated amount of time. The enzyme was then deactivated by reducing the pH to 1.9 with dilute hydrochloric acid. After 5 minutes at pH 1.9, the pH of the slurry was adjusted to a value between 5.0 and 5.5 with dilute sodium hydroxide. The reaction mixture was filtered, washed with tap water, and dried to a moisture content of 5-10%. The conditions and results are set forth in Table 1.

TABLE 1

| | | | | | | Starch Properties | | | |
|---|---|---|---|---|---|---|---|---|---|
| Reaction Conditions | | | | | | | Absorption (mL/10 g ds) | | |
| | Dosage | Temp. | | | % | Hydrophobicity/ | | 1% | |
| Enzyme | (mL) | (° C.) | pH | Time | Yield | delayed wettability | Water | saline | Oil |
| Unhydrolyzed dent | | — | — | — | — | − | 8.0 | 9.0 | 7.0 |
| corn starch (Control) | | | | | | | | | |
| G990 | 5.0 | 60 | 5.20 | 4 h | 90.5 | + | 12.0 | 11.5 | 8.5 |
| | 10.0 | 60 | 5.20 | 8 h | 78.5 | + | 14.0 | 14.0 | 9.0 |

### Comparative Example 1

Example 1 was repeated using various amylase enzymes (alpha-amylase G995, from Enzyme Bio-Systems, Ltd., and alpha-amylase BAN and beta-amylase Maltogenase 4000L, both commercially available from Novo Nordisk). The reaction conditions and properties of the resulting starches are set forth below in Table CE-1.

TABLE CE-1

| | | | | | | Starch Properties | | | |
|---|---|---|---|---|---|---|---|---|---|
| Reaction Conditions | | | | | | | Absorption (mL/10 g ds) | | |
| | Dosage | Temp. | | | % | Hydrophobicity/ | | 1% | |
| Enzyme | (mL) | (° C.) | pH | Time | Yield | delayed wettability | Water | saline | Oil |
| G995 | 0.13 | 51 | 5.80 | 24 h | 75.5 | − | 11.0 | 12.0 | 11.0 |
| BAN | 0.26 | 51 | 6.30 | 8 h | 87.1 | − | 9.5 | 10.0 | 9.5 |
| Maltogenase | 2.0 | 60 | 5.15 | 2 h | 85.7 | − | 11.0 | 11.0 | 8.5 |
| | 5.0 | 60 | 5.15 | 4 h | 77.0 | − | 11.5 | 11.5 | 11.0 |

### Example 2

This Example illustrates the various reaction conditions employed when using various glucoamylase enzymes may differ from enzyme to enzyme.

Corn starch was enzymatically hydrolyzed as discussed in Example 1 using G990 and OPTIDEX L400, a glucoamylase enzyme commercially available from Genecor International. Table 2 illustrates some of the reaction conditions and the properties resulting starches thereby obtained.

TABLE 2

| Reaction Conditions | | | | | Starch |
|---|---|---|---|---|---|
| | Dosage | Temp. | | | Hydrophobicity/ |
| Enzyme | (mL) | (° C.) | pH | Time | delayed wettability |
| G990 | 1.0 | 60° C. | 5.20 | 4 h | − |
| G990 | 2.0 | 60° C. | 5.20 | 2 h | − |
| | | | | 6 h | − |
| | | | | 8 h | − |
| | | | | 24 h | + |
| G990 | 5.0 | 43° C. | 5.20 | 4 h | − |
| G990 | 5.0 | 60° C. | 5.20 | 15 min | + |
| G990 | 10.0 | 60° C. | 5.20 | 2 h | + |
| Optidex | 0.30 | 60° C. | 4.15 | 4 h | − |
| L-400 | | | | | |
| Optidex | 3.0 | 60° C. | 4.15 | 4 h | + |
| L-400 | | | | | |

### Example 3

This Example illustrates that the invention remains operable when the starch is treated with a small amount of an alpha-amylase, with the resulting starch granules retaining their hydrophobicity and delayed aqueous wettability characteristics.

Dent corn starch was enzymatically hydrolyzed as discussed in Example 1, except that the enzyme dosage was varied as described as follows in Table 3. The following results were obtained:

TABLE 3

| Reaction Conditions | | | | | | Hydrophobicity/ |
|---|---|---|---|---|---|---|
| | Dosage | Temp. | | | | delayed |
| Enzyme | (mL) | (° C.) | PH | Time | Yield | wettability |
| G990/ | 5.0/0.65 | 60° C. | 5.20 | 2 h | 50.9 | − |
| G995 | | | | | | |
| G990/ | 5.0/0.5 | 60° C. | 5.20 | 2 h | 55.7 | − |
| G995 | | | | | | |
| G990/ | 2.5/0.25 | 60° C. | 5.20 | 1 h | 62.7 | − |
| G995 | | | | | | |
| G990/ | 10.0/0.05 | 60° C. | 5.20 | 8 h | 68.5 | + |
| G995 | | | | | | |
| G990 | 10.0 | 60° C. | 5.20 | 8 h | 78.5 | + |

### Example 4

This Example illustrates that various starches may be hydrolyzed in accordance with the invention.

Example 1 was repeated, except that instead of dent corn starch, VINAMYL II a high amylose starch available from National Starch and Chemical Co., and B850, a cross-linked starch available from Grain Processing Corporation, were enzymatically hydrolyzed with G990 glucoamylase. The following results were obtained.

TABLE 4

| Reaction Conditions | | | | | | |
|---|---|---|---|---|---|---|
| | Dosage | Temp. | | | | Hydrophobicity/ |
| Starch | (mL) | (° C.) | PH | Time | Yield | delayed wettability |
| High | 10.0 | 60° C. | 5.20 | 8 h | 88.4 | + |
| Amylose | | | | | | |
| Cross- | 10.0 | 60° C. | 5.20 | 8 h | 96.4 | + |
| Linked | | | | | | |

### Example 5

This Example illustrates that corn starch that has been hydrolyzed with a glucoamylase in accordance with the invention exhibits excellent water, oil, and 1% saline absorption properties.

A spatula rub-out test in accordance with ASTM D281-95 was performed using the starches of Example 1, yielding the results shown in Table 5.

TABLE 5

| | Absorption (mL/10 g basis) | | |
|---|---|---|---|
| Powder | Water | 1% saline | Oil |
| Glucoamylase | 12.0-14.0 | 11.5-14.0 | 8.5-9.0 |
| treated corn starches | | | |

### Comparative Example 2

A spatula rub-out test in accordance with D281-95 was performed for three commercially available baby powders, two comprising talc and one comprising pure corn starch. The following results were obtained.

TABLE CE-2

| | | Absorption (mL/10 g basis) | | |
|---|---|---|---|---|
| Powder | Manufacturer | Water | 1% saline | Oil |
| Talc | Equate | 7.0 | 8.0 | 8.5 |
| Talc | Johnson & Johnson | 8.0 | 9.0 | 10.0 |
| Pure Corn | Johnson & Johnson | 10.0 | 9.5 | 7.5 |
| Starch | | | | |

As is evident from a comparison of the data in Comparative Example 2 with that of Example 5, the porous starch granules prepared in accordance with the invention generally outperformed the commercial products. The starch granules prepared in accordance with the invention may be used as a baby powder with excellent results.

### Example 6

This Example describes physical properties of various enzymatically treated starches.

Corn starch was enzymatically hydrolyzed following the procedures discussed above with respect to Example 1, except that glucoamylase, alpha-amylase, or a combination of glucoamylase and alpha-amylase were employed. The loose bulk density (evaluated by weighing 100 ml of the starch granules) and the surface area of the starch granules (evaluated by an outside facility) were determined. The following results were obtained:

TABLE 6

| | | Density | Surface Area |
|---|---|---|---|
| Enzyme | % Yield | (g/cm³) | (m²/g) |
| None* | — | 0.62 | 0.32 |
| G995/G990* | 25 | 0.49 | 1.34 |
| G990** | 70.4 | 0.66 | 0.47 |
| G995* | 71.3 | 0.52 | 1.09 |
| G995* | 58.8 | 0.46 | 1.14 |
| G990** | 70.4 | 0.66 | 0.47 |

*= Control
**= Invention

These results demonstrate that glucoamylase treatment of granular starch does not decrease the density of the treated starch granules, and that the surface area is only slightly increased compared to the native starch. Enzymatic treatment of corn starch with alpha amylase and a combination of alpha amylase and glucoamylase does increase the surface area of the granules while lowering the density of the granules.

### Example 7

This Example illustrates the preparation of a food additive.

The starch granules prepared in accordance with the teachings of Example 1 are sprayed with an orange flavoring. The resulting granules are suitable for use in connection with a preparation of an orange-flavored food product.

### Example 8

This Example illustrates a preferred embodiment of the invention.

A slurry of starch, 35 gallons (4.3 pounds/gallon, 43% solids) was charged to a reaction vessel. The temperature of the starch slurry was maintained at 46-49° C. The pH of the slurry was adjusted from 5.90 to 5.25 using 20 mL of concentrated hydrochloric acid. Glucoamylase G990-SP (Enzyme Bio-Systems Ltd., from Aspergillus niger), 1635 grams (2.4% volume enzyme/weight of starch, 4.4 units/g starch) was poured into the slurry and the mixture was stirred for five minutes. The reaction was then terminated by quickly adjusting the pH of the reaction to 1.8 by the addition of 210 mL of concentrated HCl over a ten-minute period. The slurry was then held at the pH for 15 minutes. The pH of the slurry was then re-adjusted to 5.0 by the addition of 3000 mL of 3% sodium hydroxide. The reaction was filtered, dried and screened (120 mesh). The final product moisture was 12.3%. The protein content in the final product was 0.47%. The wet-out time is listed in Table 10 of Example 10.

### Example 9

This example illustrates the preparation of another granular hydrophobic starch.

A slurry of, 35 gallons (4.3 pounds/gallon, 43% solids) was charged to a reaction vessel. The temperature of the starch slurry was maintained at 46-49° C. The pH of the slurry was 5.97. Glucoamylase G990-SP (Enzyme Bio-Systems Ltd., from Aspergillus niger) 1635 grams (2.44% v/w, 4.4 u/g) was stirred into the mixture for five minutes. The reaction was then terminated by quickly adjusting the pH the reaction to 2.0 by the addition of 250 mL of concentrated HCl over a ten-minute period. The slurry was then held at this pH for 15 minutes. The pH of the slurry was then re-adjusted to 3.59 by the addition of 1950 mL of 3% sodium hydroxide. The reaction was filtered, dried and screened (120 mesh). The final product moisture was 5.2%. The protein content in the final product was 0.46%. The wet-out time is listed in Table 10 of Example 10.

### Example 10

The Example illustrates the preparation of yet another hydrophobic granular starch.

Unmodified starch, B200, 500 g dry solids (554 g as is) was mixed into 608.5 mL of tap water to make a 43% starch solids slurry. The mixture was heated to 48° C. The pH was adjusted from 6.1 to 3.5 by the addition of 1:1 concentrated hydrochloric acid:water. Glucoamylase, 12.0 mL (G990-SP, 4.4 u/g starch, 2.4% 0.15 v/w), was added to the slurry. After five minutes the reaction was quenched by the addition of 1:1 HCl:water to a pH of 1.75. After fifteen minutes at pH 1.75, the reaction was re-adjusted to pH 3.5 by the addition of 3% NaOH. A sample of the slurry was filtered to yield a filtrate with 2% soluble carbohydrate (˜95% reaction efficiency). The remainder of the slurry was then filtered, washed with 2×400 mL of cold tap water and dried. The dried material was screened to approximately 120 mesh particle size. The wet-out time is listed in Table 10.

TABLE 10
Wet-Out Times for Selected Samples:

| Sample | Wet-Out |
|---|---|
| B200 (unmodified, 120 mesh starch) | 16 sec. |
| Starch of Example 1, second entry | 1 min, 58 sec. |
| Example 8 | 5 min, 30 sec. |
| Example 9 | 30 min, 28 sec. |
| Example 10 | 1 hour |

The data in Table 1 show how untreated starch has a very short wet-out time, thus establishing a baseline lack of hydrophobicity. The starches of Examples 8-10 were substantially hydrophobic relative to untreated starch and to the starch of Example 1.

### Example 11

Starches were treated as per Example 10, except that the pH at enzyme addition was 5.0 and the final pH of the quenching was 5.0

The following conditions were employed, yielding the following results.

TABLE 11

| | Tem- | | | | | |
|---|---|---|---|---|---|---|
| | perature | % | Enzyme Level | | Wet out time | % |
| Example | (° C.) | Solids | Units/g | % v/w | (h) | Protein |
| 11A | 43 | 28 | 1.8 | 5 | 0.033 | 0.41 |
| | | | | | (2 min) | |
| 11B | 43 | 28 | 4.4 | 12 | 0.5 | 0.55 |
| 11C | 60 | 28 | 1.8 | 5 | 0.667 | 0.41 |
| 11D | 60 | 28 | 4.4 | 12 | >72 | 0.59 |
| 11E | 51 | 35 | 3.1 | 8.5 | 18 | |
| 11F | 43 | 43 | 1.8 | 5 | 0.011 | 0.41 |
| | | | | | (40 s) | |
| 11G | 43 | 43 | 4.4 | 12 | >72 | 0.58 |
| 11H | 60 | 43 | 1.8 | 5 | 0.023 | |
| | | | | | (83 s) | |
| 11I | 60 | 43 | 4.4 | 12 | 0.025 | 0.59 |
| | | | | | (90 s) | |

The following experiments demonstrate the relation between enzyme dosage and delayed wettability, namely that the more enzyme employed, the longer is the wet-out time and thus the greater is the delayed wettability. The data also indicates that low temperature, high solids levels, and high enzyme levels are ideal conditions for generating delayed wettability starch.

### Comparative Example 3

Unmodified starch, B200, 500 g dry solids (554 g as is) was mixed into 1250 mL of tap water to make a 28% starch solids slurry. The mixture was heated to 55° C. The pH was adjusted from 6.3 to 4.5 by the addition of 1:1 concentrated hydrochloric acid:water. Glucoamylase from Rhizopus mold, 0.19 g (Sigma, 8.8 units/g starch, 0.4% w/w), was added to the slurry. After two hourse the reaction was quenched by the addition of 1:1 HCl water solution to a pH of 1.7. After five minutes at pH 1.7, the reaction was re-adjusted to pH 5.1 by the addition of 3% NaOH. A sample of the slurry was filtered to yield a filtrate with 3.65% soluble carbohydrate (˜87% reaction efficiency). The remainder of the slurry was then filtered, washed with 2×400 mL of cold tap water and dried. The dried material was screened to approximately 120 mesh particle size. The protein content of the starch was 0.27%. The wet-out time is listed in Table CE-7 of Comparative Example 7.

### Example 12

Unmodified starch, B200, 500 g dry solids (554 g as is) was mixed into 1250 mL of tap water to make a 28% starch solids slurry. The mixture was heated to 60° C. The pH was adjusted from 6.1 to 5.0 by the addition of 1:1 concentrated hydrochloric acid:water. Glucoamylase from A. niger mold, 12 mL (Genencor Optidex L400, 8.4 units/g starch, 2.4% w/w dry starch), was added to the slurry. After five minutes the reaction was quenched by the addition of a 1:1 HCl:water solution to a pH of 1.8. After five minutes at pH 1.8, the reaction was re-adjusted to pH 5.1 by the addition of 3% NaOH. A sample of the slurry was filtered to yield a filtrate with 2.8% soluble carbohydrate (˜90% reaction efficiency). The remainder of the slurry was then filtered, washed with 2×400 mL of cold tap water and dried.

The dried material was screened to approximately 120 mesh particle size. The protein content of the starch was 0.73%. The wet-out time is listed in Table CE-6 of Comparative Example 6.

### Comparative Example 4

The experiment protocol that was used for the Example was repeated except that the G990 enzyme was de-activated prior to use. This de-activation was accomplished by lowering the pH of the enzyme with 50% acetic acid to a pH of 1.85. The de-activated enzyme was then added to the starch slurry. The reaction was allowed to proceed for 2.5 hours to maximize the potential for the enzyme to bind to the surface of the starch granule. A sample of the slurry was periodically removed and filtered to yield a filtrate with no more than 0.75% soluble carbohydrate (˜97% reaction efficiency). The remainder of the slurry was then filtered, washed with 2×400 mL of cold tap water and dried. The dried material was screened to approximately 120 mesh particle size. The protein content of the starch was 0.44%. The wet-out time is listed in Table CE-6 of Comparative Example 6.

### Example 13 and Comparative Example 5

The experimental protocol that was used for Comparative Example 11D was repeated. One half of the reaction was saved as Example 13. The other half of the reaction product was pH adjusted to pH 5.05 then treated with 1 mL of Genencor Protease 899 for 30 minutes. The reaction was then worked up as in previous examples to yield sample 1796-40-2. The protein levels for samples 1796-40-1 and 1796-40-2 were 0.49% and 0.36% respectively. The wet-out times are listed in Table CE-6 of Comparative Example 6.

### Comparative Example 6

The experimental protocol that was used for Example 11D was repeated except that the enzyme used was alpha amylase G995 from Enzyme Biosystems. The amount of enzyme used was 1.3 mL (0.26% v/W, 20.8 units enzyme/g starch). The reaction time was extended to seven hours. The soluble carbohydrate in the filtrate was 12.8%, indicating that 46% of the starch granule was hydrolyzed. The slurry was filtered, washed with 2×400 mL of cold tap water and dried. The dried material was screened to approximately 120 mesh particle size. The protein content of the starch was 0.39%. The wet-out time is listed in Table CE-6.

TABLE CE-6
Wet-Out Times for Selected Samples

| Sample | Enzyme Used | Wet-out | |
|---|---|---|---|
| B200 (unmodified, 120 | None | 16 | seconds |
| mesh starch) | | | |
| Comparative Example 3 | Rhizopus Glucoamylase | 30 | sec. |
| Comparative Example 4 | Optidex L400—Genecor | 72 | hours |
| Comparative Example 5 | De-activated G990 | 43 | seconds |
| Comparative Example 6 | G990-SP, then Protease 899 | 23 | seconds |
| Comparative Example 7 | G995—alpha amylase | 1 min, 44 sec. | |
| Example 12 | G9900-SP | 72 | hours |

The data in Table 3 shows that the hydrophobicity (as determined via wet-out time) is strongest with glucoamylase from A. niger. Glucoamylase from Rhizopus was not effective, nor was alpha amylase enzyme. The data also shows that de-activation of the enzyme prior to usage prevents the hydrophobicity imparting effect. The addition of protease after glycoamylase form A. niger also destroys the hydrophobicity effect.

### Example 14

A starch slurry, 33.4 gallons (146 pounds, 43% solids) was charged to a reaction vessel. The temperature of the starch slurry was controlled at 46-48° C. The pH of the slurry was 5.4. Glucoamylase G990-SP (Enzyme Bio-Systems Ltd., from Aspergillus niger), 1635 g (2.4% weight enzyme/weight of starch, 4.4 units/g starch) was poured into the slurry mixture and the mixture was stirred for five minutes. The reaction was then terminated by quickly adjusting the pH of the reaction to 1.55 by the addition of 265 mL of concentrated HCl. The reaction was stirred an additional 15 minutes. The pH of the slurry was then re-adjusted to 5.2 by the addition of 4.40 L of 3% sodium hydroxide solution. Hydrogen peroxide, 136 mL of a 30% active solution, was then poured into the reaction and stirred an additional 30 minutes. The mixture was then filtered, dried, and screened (120 mesh). The final product moisture was 10.0%. The protein content in the final product was 0.46%. The wet-out time of the sample was over 72 hours.

### Example 15

Hydrophobic starch, 1.0 g (120 mesh) was mixed with 0.5 g of Amoco Superla White Mineral Oil 7. After mixing for several minutes, a light yellow partially flowable power was produced. The application of this powder to human skin provided a smooth, velvet-like sensation without any residual oil or greasy feel.

### Comparative Example 2

Unmodified, screened starch (120 mesh) 1.0 g was mixed with 0.5 g of Amoco Superla White Mineral Oil 7. After mixing for several minutes, a light yellow partially flowable power was produced. The application of the power to human skin provided an oily or grease-like sensation that left oil on the skin.

### Example 16

A slurry of starch, 4 liters (507.6 g/L, ˜43% solids) was charged to a reaction vessel. Warm tap water, 1.011 L, was poured into the reaction mixture to dilute the starch slurry to a solids level of 35%. The temperature of the starch slurry was maintained at 4648° C. The pH of the slurry was adjusted from 5.96 to 5.0 using N hydrochloric acid. Glucoamylase G990-SP (Enzyme Bio-Systems Ltd., from Aspergillus niger) 48 mL grams (2.4% volume enzyme/weight of starch, 4.4 units/g starch) was poured into the reaction mixture and the mixture was stirred for five minutes. The reaction was then terminated by quickly adjusting the pH of the reaction to 1.75 by the addition of 6N HCl. Hydrogen peroxide, 4 mL of a 30% active solution, was then poured into the reaction. The mixture was held at temperature for 1.5 hours. The pH of the slurry was then re-adjusted to 5.0 by the addition of 3% sodium hydroxide. A portion of the reaction was filtered, dried and screened (120 mesh). The resulting filtrate had less than 0.5% soluble carbohydrate, indicating a reaction efficiency of >98%. The final product moisture was 10.7%. The protein content in the final product was 0.46%. The wet-out time of the sample was over 72 hours. The resulting product had no measurable enzyme activity after incubation of the sample at pH 5, 48° C. for four hours.

Thus, it is seen that hydrophobic starch granules may be prepared via the treatment of starch with a glucoamylase. The porous starch granules thus prepared are hydrophobic and are suitable for use in various applications.

While particular embodiments of the invention have been shown, it will be understood that the invention is not limited thereto since modifications may be made by those skilled in the art, particularly in light of the foregoing teachings. It is, therefore, contemplated by the appended claims to cover any such modifications as incorporate those features which constitute the essential features of these improvements within the true spirit and scope of the invention. All references cited herein are hereby incorporated by reference in their entireties

1. A method for preparing a hydrophobic granular starch, comprising:
providing an aqueous solution including a glucoamylase enzyme;
providing a starch;
allowing said glucoamylase enzyme to associate with said starch at a temperature that is less that the gelatinization temperature of said starch; and
reducing the pH of said aqueous solution to a level effective to denature said enzyme and to cause said denatured enzyme to render said starch granule hydrophobic.

2. A method according to claim 1, further comprising drying said starch granule to a moisture content of about 12% or less.

3. A method according to claim 2, further comprising grinding said dried starch.

4. A method according to claim 1, including reducing said pH when said glucoamylase enzyme has hydrolyzed said starch to a hydrolysis level of not more than 5%.

5. A method according to claim 4, including reducing said pH when said glucoamylase enzyme has hydrolyzed said starch to a level of not more than 1%.

6. A method according to claim 1, wherein the starch solids level in said slurry ranges from about 10% to about 55% by dry starch weight.

7. A method according to claim 6, the starch solids level in said slurry ranging from about 25% to about 45% by dry starch weight.

8. A method according to claim 1, said glucoamylase being present in said slurry in an amount ranging about 0.2 to about 6% by dry starch weight.

9. A method according to claim 1, said starch being a native starch.

10. A method according to claim 1, said starch being a cross-linked starch.

11. The hydrophobic granular starch prepared in accordance with claim 1.

12. A cosmetic product comprising at least one skin contacting ingredient and an amount of the starch of claim 11 effective to adsorb oil from the skin when said cosmetic product is applied to the skin.

13. The hydrophobic granular starch prepared in accordance with claim 4.

14. The hydrophobic granular starch prepared in accordance with claim 5.

15. A method according to claim 2, including reducing said pH when said glucoamylase enzyme has hydrolyzed said starch to a hydrolysis level of not more than 5%.

16. A method according to claim 3, including reducing said pH when said glucoamylase enzyme has hydrolyzed said starch to a hydrolysis level of not more than 5%.

17. A method according to claim 6, including reducing said pH when said glucoamylase enzyme has hydrolyzed said starch to a hydrolysis level of not more than 5%.

18. A method according to claim 7, including reducing said pH when said glucoamylase enzyme has hydrolyzed said starch to a hydrolysis level of not more than 5%.

19. A method according to claim 8, including reducing said pH when said glucoamylase enzyme has hydrolyzed said starch to a hydrolysis level of not more than 5%.

20. A method according to claim 9, including reducing said pH when said glucoamylase enzyme has hydrolyzed said starch to a hydrolysis level of not more than 5%.

21. A method according to claim 10, including reducing said pH when said glucoamylase enzyme has hydrolyzed said starch to a hydrolysis level of not more than 5%.

22. A method according to claim 11, including reducing said pH when said glucoamylase enzyme has hydrolyzed said starch to a hydrolysis level of not more than 5%.

23. A method according to claim 2, including reducing said pH when said glucoamylase enzyme has hydrolyzed said starch to a hydrolysis level of not more than 1%.

24. A method according to claim 3, including reducing said pH when said glucoamylase enzyme has hydrolyzed said starch to a hydrolysis level of not more than 1%.

25. A method according to claim 5, including reducing said pH when said glucoamylase enzyme has hydrolyzed said starch to a hydrolysis level of not more than 1%.

26. A method according to claim 6, including reducing said pH when said glucoamylase enzyme has hydrolyzed said starch to a hydrolysis level of not more than 1%.

27. A method according to claim 7, including reducing said pH when said glucoamylase enzyme has hydrolyzed said starch to a hydrolysis level of not more than 1%.

28. A method according to claim 8, including reducing said pH when said glucoamylase enzyme has hydrolyzed said starch to a hydrolysis level of not more than 1%.

29. A method according to claim 9, including reducing said pH when said glucoamylase enzyme has hydrolyzed said starch to a hydrolysis level of not more than 1%.

30. A method according to claim 10, including reducing said pH when said glucoamylase enzyme has hydrolyzed said starch to a hydrolysis level of not more than 1%.

31. A method according to claim 1, wherein said glucoamylase enzyme is derived from A. niger.

32. A method according to claim 4, wherein said glucoamylase enzyme is derived from A. niger.

33. A method according to claim 5, wherein said glucoamylase enzyme is derived from A. niger.
